(19) United States
(12) Patent Application Publication
(10) Pub. No.: US 2009/0230318 A1
(43) Pub. Date: Sep. 17, 2009

(54) TARGET DESIGN FOR HIGH-POWER LASER ACCELERATED IONS

(75) Inventors: Eugene S Fourkal, Philadelphia, PA (US); Iavor Veltchev, Huntington Valley, PA (US); Chang Ming Ma, Huntington Valley, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Fox Chase Cancer Center, Philadelphia, PA (US)

(21) Appl. No.: 11/720,886

(22) PCT Filed: Dec. 22, 2005
(86) PCT No.: PCT/US05/46838

§ 371 Date: Jun. 5, 2007

Related U.S. Application Data
(60) Provisional application No. 60/638,821, filed on Dec. 22, 2004.

(51) Int. Cl. H01J 27/00; G21K 1/00; A61N 5/00

(52) U.S. Cl. 250/423 R; 250/505.1; 250/492.3

(57) ABSTRACT

Methods for designing a laser-accelerated ion beam are disclosed. The methods include modeling a system including a heavy ion layer, an electric field, and high energy light positive ions having a maximum light positive ion energy, correlating physical parameters of the heavy ion layer, the electric field, and the maximum light positive ion energy using the model, and varying the parameters of the heavy ion layer to optimize the energy distribution of the high energy light positive ions. One method includes analyzing the acceleration of light positive ions, for example protons, through interaction of a high-power laser pulse with a double-layer target using two-dimensional particle-in-cell (PIC) simulations and a one-dimensional analytical model. The maximum energy acquired by the accelerated light positive ions, e.g., protons, in this model depends on the physical characteristics of the heavy-ion layer—the electron-ion mass ratio and effective charge state of the ions. The hydrodynamic equations for both electron and heavy ion species solved and the test-particle approximation for the protons is applied. It was found that the heavy ion motion modifies the longitudinal electric field distribution, thus changing the acceleration conditions for the light positive ions.

### CROSS REFERENCE TO RELATED APPLICATIONS

This patent application claims the benefit of U.S. Provisional Patent Application Ser. No. 60/638,821, filed Dec. 22, 2004, the entirety of which is incorporated by reference herein.

### STATEMENT OF GOVERNMENT SUPPORT

This work is partly supported by the Department of Health and Human Services, the National Institute of Health, under the contract number CA78331. Accordingly, the Government may have rights in these inventions.

### FIELD OF THE INVENTION

The field of the invention pertains to laser-accelerated light positive ions, such as protons, generated from the interaction of ultrahigh intensity laser pulses and target materials. The field of the invention also pertains to targets and their design for interacting with ultrahigh intensity laser pulses for generating high energy light positive ions.

### BACKGROUND OF THE INVENTION

The interaction of ultrahigh intensity laser pulses with plasmas has attracted considerable interest due to its promising applications in a variety of areas such as generation of hard X-rays, neutrons, electrons, and high energy ions. The laser-accelerated ion beams have specific characteristics, such as high collimation and high particle flux, which make them very attractive for applications in controlled nuclear fusion, material science, production of short-lived isotopes for medical diagnostics, and hadron therapy (e.g., proton beam radiation for the treatment of cancer).

There is presently a need to create target materials that can controllably provide ion beams of controlled composition and energy distribution. Previous experimental studies have been directed toward the understanding of different mechanisms of fast proton/ion generation during the interaction of ultrahigh intensity laser pulses with thin solid structures (i.e., targets) Metallic as well as insulator targets were used with a thickness ranging from a few microns “μm” to more than 100 μm. The origin of the observed ions and the mechanism of their acceleration still remain matters of debate. The ions are either created and accelerated at the front surface directly illuminated by the incident laser, or at the rear surface, where the acceleration occurs through the electrostatic field, generated by the space-charge separation. The particular experimental conditions (the influence of the laser pedestal and the target properties) can determine the acceleration scheme, although in some experiments it has been shown that the proton acceleration occurs at the back surface of the target. Accordingly, there is a need to better understand the dynamics of the interaction of intense laser pulses with materials. This understanding will, in turn, give rise to improved target designs and methodologies for designing targets for generating laser accelerated ion beams.

One theoretical model for ion acceleration at the back surface of the target is based on quasi-neutral plasma expansion into vacuum. In this model, the accelerating electric field is generated due to space-charge separation in a narrow layer at the front of the expanding plasma cloud, which is assumed to be neutral. In the interaction of an ultrashort and ultraintense laser pulse with a solid structure, the assumption of quasi-neutrality is abandoned. The results of computer simulations suggest that the interaction of petawatt laser pulses with plasma foils leads to the formation of extended regions where plasma quasi-neutrality is violated, a factor that should be taken into account when considering ion acceleration by ultraintense pulses. Passoni et al., Phys. Rev. E 69, 026411 (2004) describes the electric field structure created by two populations of electrons, each following Boltzmann distribution with different thermal energies. The effects of charge separation have been taken into account by solving Poisson equations (with two-temperature electron components) for the electrostatic potential distribution inside the foil (where ions are present) and outside of it (where electrons reside). This approach is limited because it inherently provides a time-independent description. However, for estimating ion energies quantitatively, the temporal evolution (i.e., time-dependent) of the electric field profile needs to be known. Although the treatment suggested by S. V. Bulanov, et al., Plasma Phys. Rep. 30, 21 (2004) offers a possibility for obtaining the spatio-temporal evolution of the self-consistent electrostatic field, further work is needed for understanding and estimating the maximum energy that ions can acquire in the field. As well, further work is needed for designing and optimizing laser-accelerated ion beam systems that are capable of generating positive ions having energy distributions that are useful in medical applications.

There are several theoretical examples of proton/ion acceleration under the condition of strong charge separation. One is the Coulomb explosion of an ion cluster. A laser pulse interacting with the target expels electrons, thus creating a strong electric field inside the foil, which plays a key role in the ion acceleration process. In other cases, protons are accelerated by the electric field (time-independent) of the ionized target and their dynamics can be described by using the test-particle approximation approach. The multi layer target system, and more specifically the two-layer one, has a particularly good structure for this acceleration scheme. In this structure the first layer has heavy ions of mass mᵢ and specific ionization state Zᵢ and the second layer (attached to its back surface) has ionized hydrogen. Under the action of the laser ponderomotive force, electrons escape from the target, leaving behind a charged layer of heavy ions. If the ion mass is much larger than that of the proton, the dynamics of the ion cluster (Coulomb explosion) is usually neglected during the effective acceleration time of protons. During this time period, the electric field of the ion cluster is considered to be time-independent and one is left with the problem of proton acceleration in a stationary, but spatially inhomogeneous electric field.

Although the aforementioned work is useful for describing ion acceleration dynamics, the proton acceleration time is actually relatively long (t≈100/ωₚₑ) and the influence f both the self-consistent electron dynamics and the ion cluster explosion typical result in the electric field being time-dependent. As a result, the maximum proton energy typically depends on the physical properties of the cluster (e.g., ion mass and charge state). Accordingly, the influence of a cluster's characteristics on the accelerating electric field and the maximum proton energy of laser interaction with a double-layer target are not fully understood. Thus, there is presently a need to better understand the interaction of high energy laser pulses with target materials for designing improved targets. This understanding will, in turn, give rise to improved target designs and methodologies for designing targets for generating laser accelerated ion beams.

### SUMMARY OF THE INVENTION

The present invention provides a model of electric field evolution that accounts for the influence of the Coulomb explosion effect. This model is used to design targets and laser-accelerated ion beams comprising high energy light ions. As used herein the term “high energy” refers to ion beams having energies in the range of from about 50 MeV to about 250 MeV. The model is based on the solution of one dimensional hydrodynamic equations for electron and ion components. The results obtained within the realm of this model are used to correlate the physical parameters of a heavy ion layer in a target with the structure of the electric field and the maximum proton energy. These results give rise to design equations for designing double-layer targets that are useful for generating high energy light positive ions, such as protons.

The present invention further provides methods for designing targets used for generating laser-accelerated ion beams. These methods typically comprise modeling a system including a heavy ion layer, an electric field, and high energy protons having an energy distribution comprising a maximum proton energy, correlating physical parameters of the heavy ion layer, the electric field, and the maximum proton energy using the model, and varying the parameters of the heavy ion layer to optimize the energy distribution of the high energy protons.

The present invention also provides methods for designing targets used for generating laser-accelerated ion beams and targets made in accordance with such methods, comprising modeling a system including a target comprising a heavy ion layer, an electric field, and high energy protons having an energy distribution comprising a maximum proton energy, wherein the system capable of being described by parameter χ, and varying the parameter χ to optimize the energy distribution of the high energy protons.

The present invention also provides methods for designing a laser-accelerated ion beam, comprising: modeling a system including a heavy ion layer, an electric field, and high energy light positive ions having an energy distribution comprising a maximum light positive ion energy; correlating physical parameters of the heavy ion layer, the electric field, and the maximum light positive ion energy using said model; and varying the parameters of the heavy ion layer to optimize the energy distribution of the high energy light positive ions.

The present invention also provides methods for designing a target used for generating laser-accelerated ion beams, comprising: modeling a system including a target, an electric field, and high energy light positive ions having an energy distribution comprising a maximum light positive ion energy, said target comprising a heavy ion layer characterized by a parameter χ; and varying the parameter χ to optimize the energy distribution of the high energy light positive ions.

The present invention also provides targets for use in generating laser-accelerated high energy light positive ion beams in a system, the targets made by the process of: modeling a system including the target, an electric field, and high energy light positive ions having an energy distribution comprising a maximum light positive ion energy, said target comprising a heavy ion layer characterized by a parameter χ; and varying the parameter χ to optimize the energy distribution of the high energy light positive ions.

The present invention also provides targets used for generating laser-accelerated ion beams in a system including the target, an electric field, and high energy light positive ions having an energy distribution comprising a maximum light positive ion energy, said target comprising: a heavy ion layer characterized by a parameter χ, wherein varying the parameter χ maximizes the energy distribution of the high energy light positive ions of the modeled system.

These and other aspects of the present invention will be readily be apparent to those skilled in the art in view of the following drawings and detailed description. The summary and the following detailed description are not to be considered restriction of the invention as defined in the appended claims and serve only to provide examples and explanations of the invention.

### BRIEF DESCRIPTION OF THE DRAWINGS

The foregoing summary, as well as the following detailed description, is further understood when read in conjunction with the appended drawings. For the purpose of illustrating the invention, there is shown in the drawings exemplary embodiments of the invention; however, the invention is not limited to the specific methods, compositions, and devices disclosed. In the drawings:

FIG. 1 is a schematic diagram of an embodiment of the laser-target system, in which the target consists of a high-density heavy ion slab with low density hydrogen layer attached to its back surface;

FIG. 2 depicts the distribution of (a) the longitudinal (Eₓ) and (b) the transverse (E_{y}) components of the electric field in the (x, y) plane at t=40/ωₚₑ. ωₚₑ≈3.57×10¹⁴ s⁻¹.

FIG. 3 depicts the energy distributions of (a) electrons, (b) protons, and (c) heavy ions at t=32/ωₚₑ for three different values of the structural parameter χ.

FIG. 4 depicts the spatial distributions of the (a) electron, (b) proton, and platinum-ion densities in the (x, y) plane at t=32/ωₚₑ, ωₚₑ≈3.57×10¹⁴ s⁻¹.

FIG. 5 depicts the longitudinal electric field profile Eₓ(x, L_{y}/2) as a function of x at t=32/ωₚₑ for three different ion-to-proton mass ratios and the same ionization state Zᵢ=4, ωₚₑ≈3.5×10¹⁴ s⁻¹.

FIG. 6 depicts the electron phase space distribution (a) and density distributions (b) for electrons (solid line) and ions (dotted line) at =150/ωₚₑ. The initial electron momentum distribution p_{e,0}=10mₑc for (0<x<½) and p_{e,0}=−10mₑc for (−½<x<0).

FIG. 7 depicts the numerically obtained parameter γ approximated by the simple expression γ(\tilde{p}_{e,0})=(1+a\tilde{p}_{c,0}²)^{b}, where a=0.691(4), b=0.2481(2), and \tilde{p}_{e,0} is the normalized electron initial momentum.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

In one aspect of the present invention, the influence of the cluster's characteristics on the accelerating electric field and the maximum proton energy using particle-in-cell (PIC) simulations of laser interaction with a double-layer target is determined. A theoretical model of electric field evolution that accounts for the influence of the Coulomb explosion effect is provided. This model is based on the solution of one dimensional hydrodynamic equations for electron and ion components. The results obtained within the realm of this model explain the correlation between the physical parameters of the heavy ion layer on one hand and the structure of the electric field and maximum proton energy on the other.

Computer Simulation Results. A two dimensional PIC numerical simulation code was used to describe the interaction of a high-power laser pulse with a double-layer target. The PIC simulation reveals the characteristic features of laser interaction with plasmas, specifically in cases where the contribution of nonlinear and kinetic effects makes the multidimensional analytical approach extremely difficult. Acceleration of protons is considered in the interaction of laser pulse with a double-layer target. The calculations were performed in a 2048×512 simulation box with a grid size Δ=0.04 μm and total number of simulated quasi-particles 4×10⁶. Periodic boundary conditions for particles and electromagnetic fields have been used. In order to minimize the influence of the boundary conditions on the outcome of the simulations, the maximum simulation time was set to 80/ωₚₑ≈225 fs, where ωₚₑ is the electron plasma frequency. Several types of targets with different electron-to-ion mass ratios and ionization states have been investigated. The ionization state of ions can be calculated from the solution to the wave equation for a given multi-electron system in the presence of an ultra-high intensity laser pulse. As calculating the ionization state is commonly tedious in systems with two or more electrons, the ion charge state can be provided in some embodiments as a parameter rather than a calculated value.

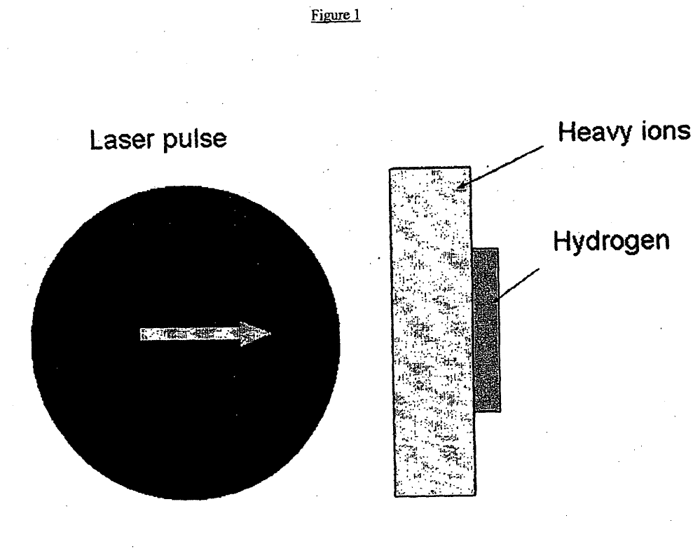

FIG. 1 shows a schematic diagram of an embodiment of the double-layer target. One embodiment can include a 0.4 μm-thick high-density (nₑ≈6.4×10²² cm⁻³) heavy-ion foil with a 0.16 μm-thick low density (nₑ≈2.8×10²⁰ cm⁻³) hydrogen layer attached to its back surface. The target was positioned in the middle of the simulation box with the laser pulse entering the interaction region from the left. The electric field of the laser pulse is polarized along the y axis with a dimensionless amplitude a=eE₀/mₑωc=30, which corresponds to the laser peak intensity of 1.9×10²¹ W/cm² for a laser wavelength of λ=0.8 μm. The laser pulse was Gaussian in shape with length (duration) and width (beam diameter) of 15λ and 8λ (FWHM), respectively, which corresponds to approximately a 890-TW system.

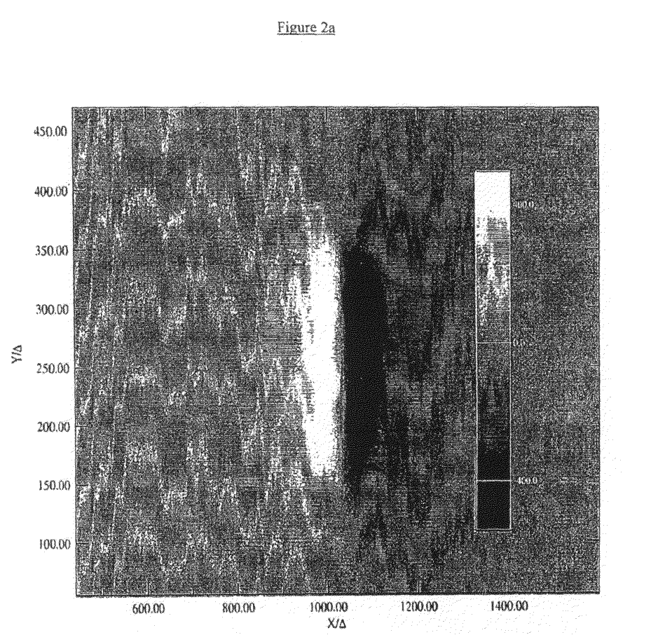

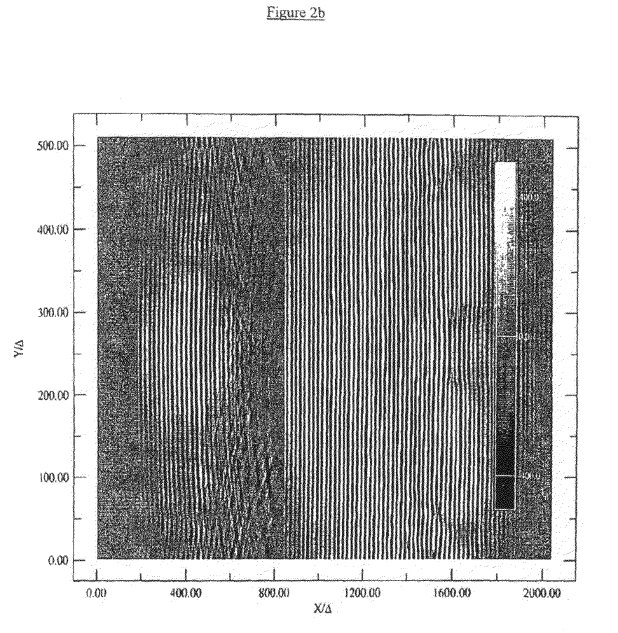

In FIG. 2 the spatial distribution of Eₓ (longitudinal) and E_{y} (transverse) components of the electric field is presented at t=40/ωₚₑ. Even though the target thickness is much larger than the collisionless skin depth, the incident pulse splits into reflected and transmitted components due to the relativistic decrease of the electron plasma frequency. As a result, a part of the laser energy goes through the overcritical density target. The longitudinal electric field, which accelerates protons, extends over large spatial distances on both sides of the target. This field is created by the expanding electron cloud accelerated in forward and backward directions by the propagating laser pulse.

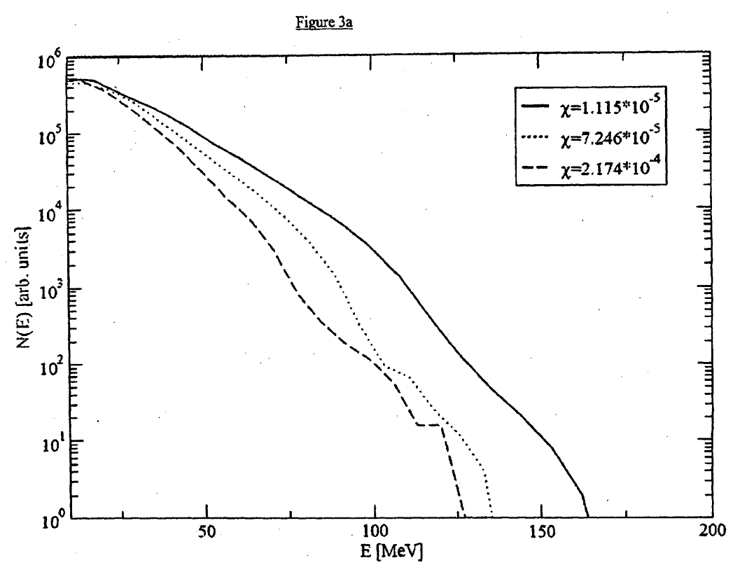

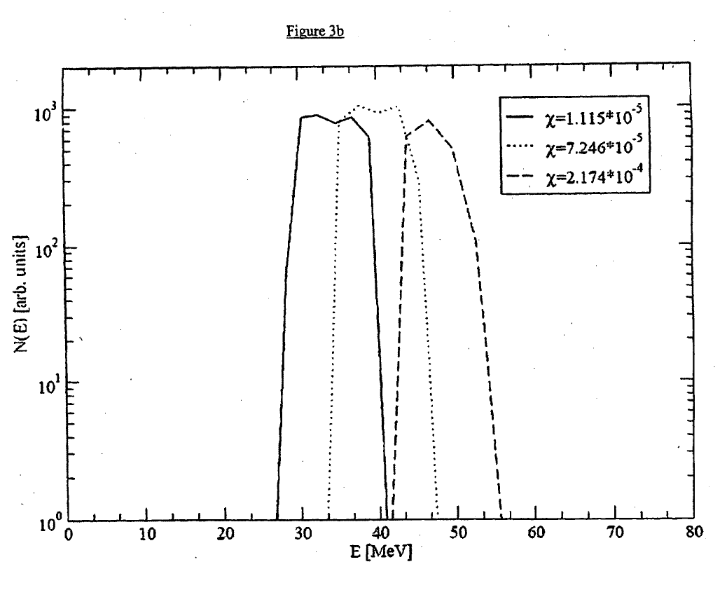

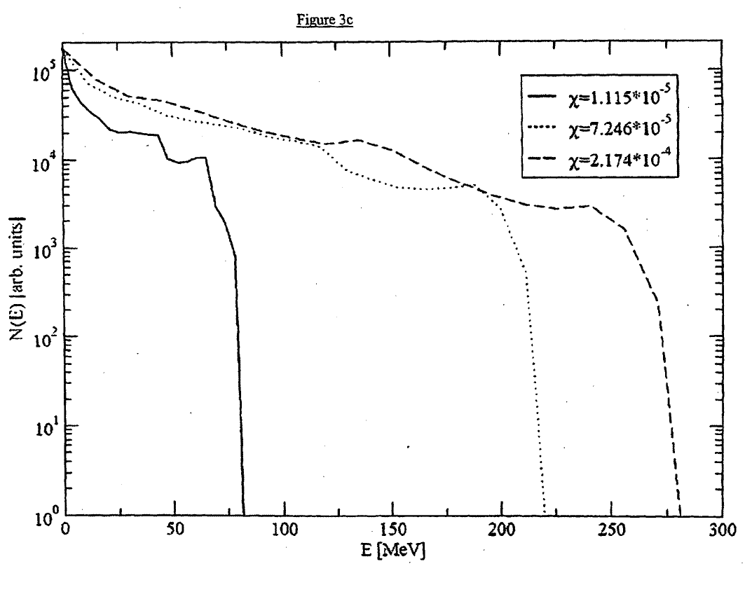

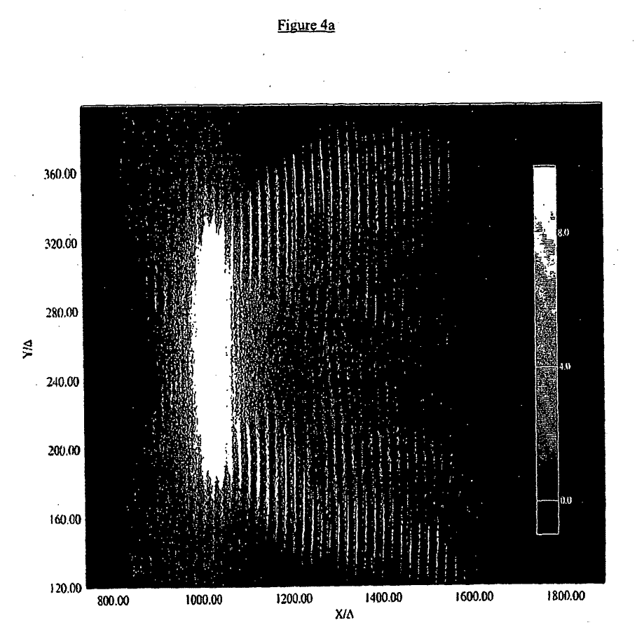

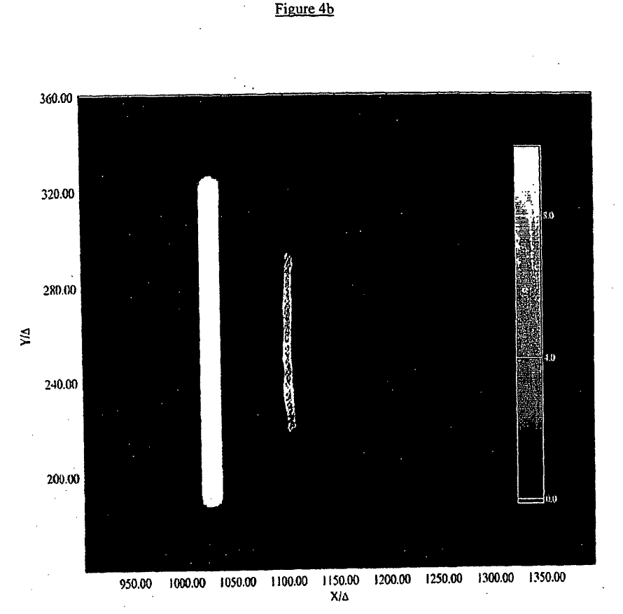

FIG. 3 shows the energy distributions of (a) electrons, (b) protons, and (c) heavy ions at t=32/ωₚₑ for different values of the structural parameter of the substrate χ=Zᵢmₑ/mᵢ. It can be seen that the electron and heavy ion energy spectra resemble quasi-thermal distributions whereas the proton energy spectrum has a quasi-monoenergetic shape with a characteristic energy depending on the value of χ. T. Z. Esirkepov, S. V., et al., Phys. Rev. Lett: 89, 175003 (2002) shows that a high quality proton beam can be generated from a double layer target geometry. When a laser pulse interacts with the target, both the heavy atoms in the first layer and the hydrogen atoms in the second are ionized; a plasma sandwich structure is thus created, consisting of the high-Z heavy ion plasma and the ionized hydrogen “attached” to its back surface. Under the action of the ponderomotive force, some electrons are expelled from the plasma (in forward and backward directions), thus producing a longitudinal electric field that accelerates the thin layer until it is sufficiently small the longitudinal electric field is not significantly perturbed. Under this condition, the protons are accelerated by the electric field created between the charged heavy-ion layer and the fast electron cloud. In this embodiment, a thinner proton layer results in narrower energy spread of the accelerated protons. Without being bound by a particular theory of operation, this is due to the fact that at any given time the protons in a narrow slab experience almost the same accelerating electric field. This peculiarity in the proton dynamics can also be seen from the spatial distributions of the particles shown in FIG. 4 for (a) electron, (b) proton and platinum-ion ( Zᵢ=4, mᵢ/mₚ=195 ) densities in (x, y) plane. At time t=32/ωₚₑ the proton layer is already detached from the high- Z target and travels almost undistorted in a forward direction. At the same time, the heavy ion layer is expanding at a much slower rate due to its greater mass. The characteristic response time of ions is on the order of ion plasma frequency 1/ωₚᵢ=\sqrt{mᵢ/4πe²n₀Zᵢ²}, where n₀ is the ion density. Once the electrons have left the target, the ion layer begins to expand under the action of the Coulomb repulsive forces. Even though the ion response time is longer than that of protons, its dynamics appear to influence the longitudinal electric field, thus affecting the acceleration of the proton beam.

As one can see from FIG. 3, larger values of the parameter x=Zᵢmₑ/mᵢ results in more effective proton acceleration (nearly 50% increase for carbon substrate compared to platinum one, assuming the same ionization state Zᵢ=4). In other words, more robust ion expansion leads to a more efficient proton acceleration. At first, this result seems somewhat counterintuitive since ion expansion is accompanied by the reduction of the longitudinal electric field (electric field energy partly transforms into the kinetic energy of the expanding ions) and can presumably lead to lower proton energies.

A simple estimation of the maximum proton energy can be ascertained from the picture suggested by S. V. Balanov, et al., Plasma Phys. Rep. 28, 975 (2002) where the longitudinal electric field of the charged layer of heavy ions is approximated by that created by a charged ellipsoid with its major semi-axis equal to the transverse dimension of the target R₀ and its minor semi-axis equal to I (2I is the thickness of a target). In this case the longitudinal electric field and the electrostatic potential have the following forms (Landau and Lifshits, Electrodynamics of Continuous Media, Pergamon Press, Oxford, 1988),

$$\begin{matrix}{E}_{x} ( x ) = \frac{8 π {en}_{0} {Z}_{i} {lR}_{0}^{2}}{3} \frac{1}{( {R}_{0}^{2} - {l}^{2} + {x}^{2} )} & ( 1 ) \\ Φ ( x ) = \frac{4 π {en}_{0} {Z}_{i} {lR}_{0}^{2}}{3} \frac{1}{( {R}_{0}^{2} - {l}^{2} )} arc tan [ \frac{\sqrt{{R}_{0}^{2} - {l}^{2}}}{x} ] & ( 2 )\end{matrix}$$

The maximum kinetic energy that a proton acquires in this field can be equal to its potential energy at the surface of the target. Under the assumption that the target thickness is much less than its transverse dimension one obtains,

ε≈2πZᵢe²n₀1R₀ (3)

In one embodiment, the estimation in Eqn (3) gives an upper limit to the maximum proton energy, which can be determined by assuming that all electrons escape from the target acquiring enough kinetic energy to overcome the attractive electric field, so that they never return to the target. In reality, however, for the laser intensity used in the simulations, typically a small fraction of electrons escape the target. The rest remain in the vicinity of the target with some of them performing a rather complicated oscillatory motion (see below). This effect greatly reduces the total charge density in the foil, thus substantially lowering the maximum proton energy estimated by Eqn (3). Eqn (3) apparently does not explain the dependence of proton energy on the ion mass and ionization state of the foil (for a given initial electron density). The combination of both the Coulomb explosion of the target and the electron dynamics in a self-consistent electric field renders the field time-dependent in contrast with the simplified model offered by Eqn (1).

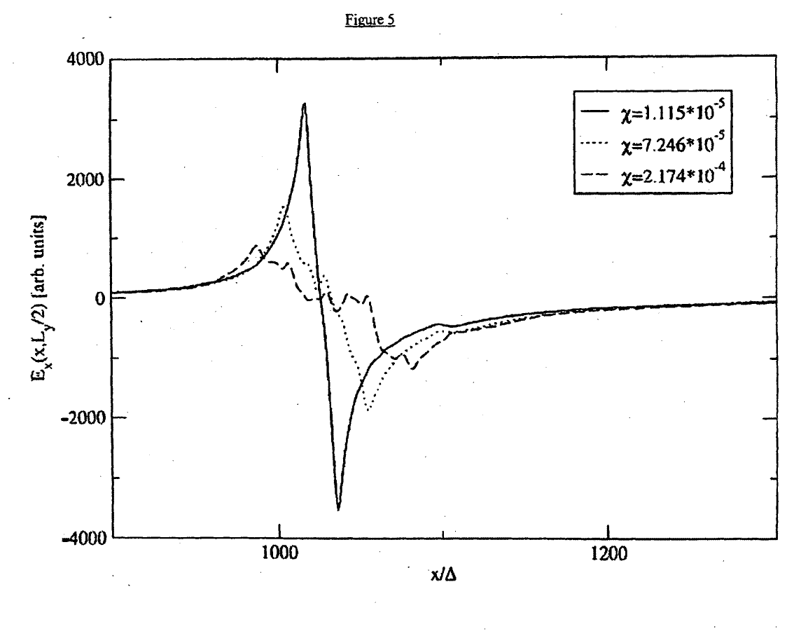

The dependence of the maximum proton energy on the target parameters typically come from the influence of the ion motion on the longitudinal electric field. FIG. 5 shows the electric field profile as a function of the distance from the target in the longitudinal direction, the direction of proton acceleration, at t=32/ωₚₑ for three different ion-to-proton mass ratios, having the same ionization state of Zᵢ=4. The electric field structure is such that its magnitude at the surface of the expanding heavy-ion layer (the point where the electric field starts decreasing with distance) increases with the ion mass because of the less efficient conversion of the field energy into kinetic energy of ions. On the other hand, further away from the target the electric field exhibits an opposite trend in which its value decreases with increasing ion-to-proton mass ratio. Since a layer of protons quickly leaves the surface of the target (before any significant target expansion occurs), the field distribution beyond the foil typically determines the maximum proton energy.

The problem of proton acceleration in the self-consistent electric field created by the expanding electron and heavy ion clouds can also be considered in one embodiment. Also, the influence of the Coulomb explosion effect on the structure of the accelerating electric field can also be evaluated in this and other embodiments. Since the interaction of a high-intensity laser pulse with plasma constitutes an extremely complicated physical phenomenon, a somewhat simplified physical picture can be considered that allows certain aspects related to the evolution of the longitudinal electric field to be clarified.

Electrons are presumed to be initially located inside the target with a flat density distribution n_{c}=Zᵢn₀θ(½−|x|), where n_{e,0}=Zᵢn₀ and θ(x) is the Heaviside unit-step function. Under the action of a high-intensity short laser pulse, the electrons typically gain the longitudinal relativistic momentum p_{e,0}. This momentum can be a function of the initial electron position xᵢ(0). A model can be provided, in which half of the electrons (located in the interval 0<x<½) gains momentum p_{e,0} from the laser pulse and the other half (located in the interval −½<x<0) gains negative momentum −p_{e,0}. This model can be somewhat descriptive of the electron fluid motion due to its interaction with the laser pulse where the forward moving particles correspond to those that are accelerated by the ponderomotive force, while the backward moving electrons are extracted in the opposite direction due to the process known as “vacuum heating”. Although this model constitutes a considerable simplification in the description of the initial electron fluid momentum distribution, it can properly describe the relevant physical mechanisms of electric field evolution.

A. Self-consistent evolution of electron cloud. The expansion of plasma into the vacuum can be described by using one-dimensional hydrodynamic equations for electron and ion components. In one embodiment, it can be assumed that the proton layer does not perturb the generated electric field. In this case the equations of hydrodynamics for both components are:

$$\begin{matrix}\frac{\partial {n}_{e}}{\partial t} + \frac{\partial ( {n}_{e} {υ}_{e} )}{\partial x} = 0 & ( 4 a ) \\ \frac{\partial {p}_{e}}{\partial t} + {υ}_{e} \frac{\partial {p}_{e}}{\partial x} = - eE ( x , t ) & ( 4 b ) \\ \frac{\partial {n}_{i}}{\partial t} + \frac{\partial ( {n}_{i} {υ}_{i} )}{\partial x} = 0 & ( 4 c ) \\ \frac{\partial {υ}_{i}}{\partial t} + {υ}_{i} \frac{\partial {υ}_{i}}{\partial x} = \frac{{Z}_{i} e}{{m}_{i}} E ( x , t ) & ( 4 d ) \\ \frac{\partial E}{\partial x} = 4 π e [ {Z}_{i} {n}_{i} ( x , t ) - {n}_{e} ( x , t ) ] , & ( 4 e )\end{matrix}$$

where nₑ and nᵢ are the electron and ion densities, uₑ and pₑ are the electron velocity and momentum related through the expression v_{c}=cpₑ/(mₑ²c²+pₑ²)^{1/2}. In Eqn (7), below, non-relativistic ion kinematics can be used during the course of the Coulomb explosion.

In order to solve Eqs. (4), the Euler variables (x, t) can be switched to those of the Lagrange (x₀, t), where x₀ is the electron fluid element coordinate at t=0. Both sets of coordinates can be related through the following expression:

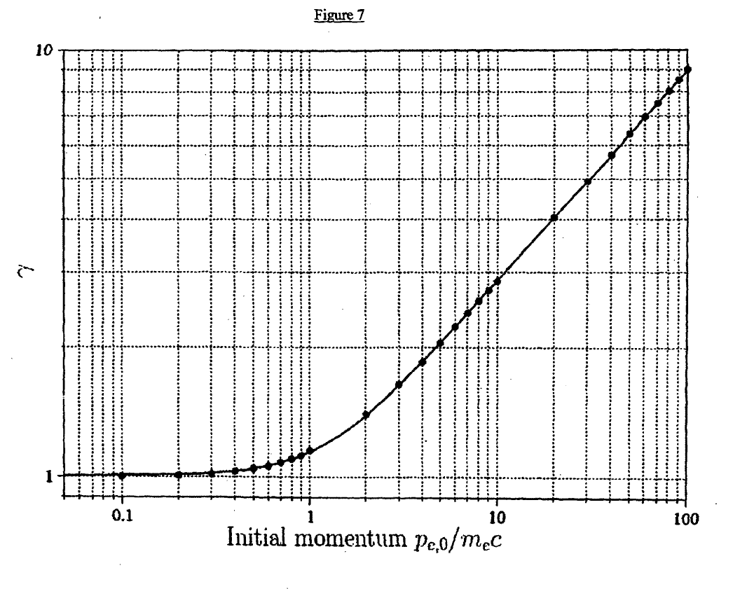

x(x₀, t)=x₀+ξₑ(x₀, t), (5)

where ξₑ(x₀, t) is the displacement of the electron fluid element from its initial position x₀ at time t. In the new variables Eqs.(4) read:

$$\begin{matrix}{\tilde{n}}_{e} ( {x}_{0} , t ) = {n}_{e} ( x , t ) = {\tilde{n}}_{e} ( {x}_{0} , 0 ) \frac{\partial {x}_{0}}{\partial x} & ( 6 a ) \\ \frac{\partial {\tilde{p}}_{e} ( {x}_{0} , t )}{\partial t} = - e \tilde{E} ( {x}_{0} , t ) & ( 6 b ) \\ \frac{\partial {\tilde{n}}_{i}}{\partial t} - {υ}_{e} \frac{\partial {x}_{0}}{\partial x} \frac{\partial {\tilde{n}}_{i}}{\partial {x}_{0}} + \frac{\partial ( {\tilde{n}}_{i} {υ}_{i} )}{\partial {x}_{0}} \frac{\partial {x}_{0}}{\partial x} = 0 & ( 6 c ) \\ \frac{\partial {v}_{i}}{\partial t} + ( {υ}_{e} - {υ}_{i} ) \frac{\partial {υ}_{i}}{\partial {x}_{0}} \frac{\partial {x}_{0}}{\partial x} = \frac{{Z}_{i} e}{{m}_{i}} \tilde{E} ( {x}_{0} , t ) & ( 6 d ) \\ \frac{\partial \tilde{E}}{\partial {x}_{0}} \frac{\partial {x}_{0}}{\partial x} = 4 π e ( {Z}_{i} {\tilde{n}}_{i} ( {x}_{0} , t ) - {\tilde{n}}_{e} ( {x}_{0} , 0 ) \frac{\partial {x}_{0}}{\partial x} ) , & ( 6 e )\end{matrix}$$

where the tilde sign is used to designate functions in the new variables (x₀, t); vₑ=∂ξₑ/∂t and vᵢ are the electron and ion fluid velocities, and ñₑ(x₀, 0)=nₑ(x, 0) is the initial electron density. The form of the hydrodynamic equations for the electron fluid component can be greatly simplified in the new variables, whereas the equations for the ions can be somewhat more complex compared to those expressed through variables (x, t). Because of the smallness parameter χ=Zᵢmₑ/mᵢ<<1, the ion motion in Eqs. (6) can be considered a perturbation to the zeroth order solution, which corresponds to the case of motionless ions. solutions to Eqs. (6) with vₑ=0 and ñᵢ(x₀, t)=n(x, 0)=n₀θ(½−|x|) for a case of constant initial electron momentum distribution can be given by the following expressions,

$$\begin{matrix}\tilde{E} ( {x}_{0} , t ) = - 4 π {eZ}_{i} {n}_{0} \{ \begin{matrix}\frac{l}{2} - {x}_{0} , & \frac{l}{2} < {x}_{0} + {ξ}_{e} \\ {ξ}_{e} ( {x}_{0} , t ) , & \vert {x}_{0} + {ξ}_{e} \vert < \frac{l}{2} \\ - \frac{l}{2} - {x}_{0} , & {x}_{0} + {ξ}_{e} < - \frac{l}{2}\end{matrix} & ( 7 ) \\ {p}_{e} ( {x}_{0} , t ) \approx \{ \begin{matrix}{p}_{e , 0} cos ( \frac{{ω}_{pe} t}{γ} ) , & t \leq {τ}^{*} , & 0 < {x}_{0} + {ξ}_{e} < \frac{l}{2} \\ {p}_{e , 0} cos ( \frac{( \frac{l}{2} - {x}_{0} ) {ω}_{pe}}{{υ}_{e , 0} γ} ) + \frac{κ ( {x}_{0} )}{{υ}_{e , 0}} ( \begin{matrix}\begin{matrix}\frac{l}{2} - \\ {x}_{0} -\end{matrix} \\ {υ}_{e , 0} t\end{matrix} ) , & t > {τ}^{*} , & {x}_{0} + {ξ}_{e} > \frac{l}{2}\end{matrix} & ( 8 a ) \\ {ξ}_{e} ( {x}_{0} , t ) \approx \{ \begin{matrix}γ \frac{c}{{ω}_{pe}} arc tan [ \frac{{p}_{e , 0} sin ( \frac{{ω}_{pe} t}{γ} )}{\sqrt{{m}_{e}^{2} {c}^{2} + {p}_{e , 0}^{2} {cos}^{2} ( \frac{{ω}_{pe} t}{γ} )}} ] , & t \leq {τ}^{*} \\ \begin{matrix}( \frac{l}{2} - {x}_{0} ) + \frac{c}{κ ( {x}_{0} )} ( \sqrt{{m}_{e}^{2} {c}^{2} + {p}_{e , 0}^{2} {cos}^{2} ( \frac{( \frac{l}{2} - {x}_{0} ) {ω}_{pe}}{{υ}_{e , 0} γ} ) -} \\ \sqrt{\begin{matrix}{m}_{e}^{2} {c}^{2} + [ {p}_{e , 0} cos ( \frac{( \frac{l}{2} - {x}_{0} ) {ω}_{pe}}{{υ}_{e , 0} γ} ) + \\ {\frac{κ ( {x}_{0} )}{{υ}_{e , 0}} ( \frac{l}{2} - {x}_{0} - {υ}_{e , 0} t ) ]}^{2}\end{matrix}} ) ,\end{matrix} & t > {τ}^{*}\end{matrix} \\ κ ( {x}_{0} ) = 4 π {Z}_{i} {e}^{2} {n}_{0} ( \frac{l}{2} - {x}_{0} ) , & ( 8 b )\end{matrix}$$

where \dot{τ}≈(½−x₀)/v_{e,0} (v_{e,0}≈c) is the transit time during which electrons are inside the target (0<x<½) and γ(p_{e,0}) is a parameter that can depend on the initial electron momentum p_{e,0}. Its value can be found from the numerical solution of Eqn (6b) for the case when electrons are inside the target and its simple analytical form γ(p_{e,0})=(1+a(p_{e,0}/mₑc)²)^{b} is shown in FIG. 7. Eqs. (8) describe the electrons that can satisfy the following condition:

$$γ ( {p}_{e , 0} ) \frac{c}{{ω}_{pe}} arc tan [ \frac{{p}_{e , 0}}{{m}_{e} c} ] > \frac{l}{2} - {x}_{0} ,$$

which provides that an electron reaches the boundary of the target (some electrons that are initially located deeply inside the target may not reach its surface). Eqs. (8a-8b) are somewhat different from those published by Bulanov, et al. due to accounting for the finite time required for electrons to leave the target. At time

$${t}_{max} = \frac{{p}_{e , 0}}{κ ( {x}_{0} )} cos [ \frac{( \frac{l}{2} - {x}_{0} ) {ω}_{pe}}{{υ}_{e , 0} γ} ] + \frac{\frac{l}{2} - {x}_{0}}{{υ}_{e , 0}}$$

the electron fluid displacement reaches the maximum value:

$${ξ}_{max} = ( \frac{l}{2} - {x}_{0} ) + \frac{c}{κ ( {x}_{0} )} \{ \sqrt{{m}_{e}^{2} {c}^{2} + {p}_{e , 0}^{2} {cos}^{2} [ \frac{( \frac{l}{2} - {x}_{0} ) {ω}_{pe}}{{υ}_{e , 0} γ} ]} - {m}_{e} c \}$$

and decreases afterwards. Eventually the electron fluid element returns to the target and reappears on the other side.

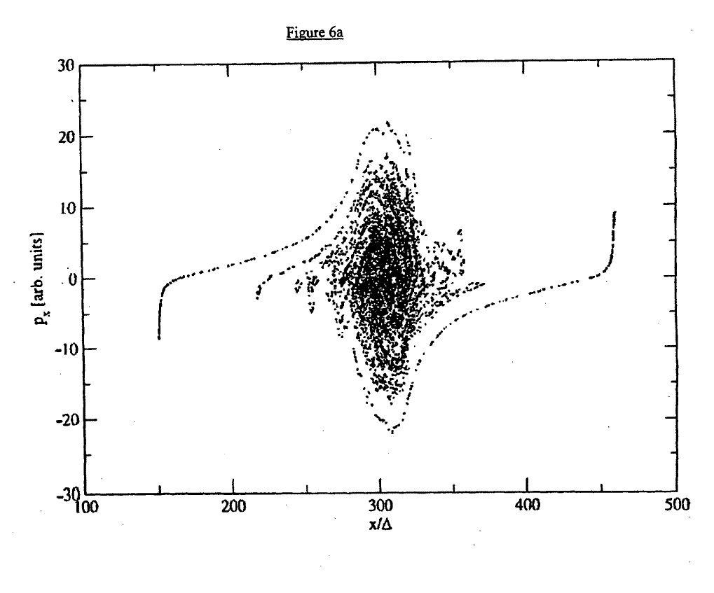

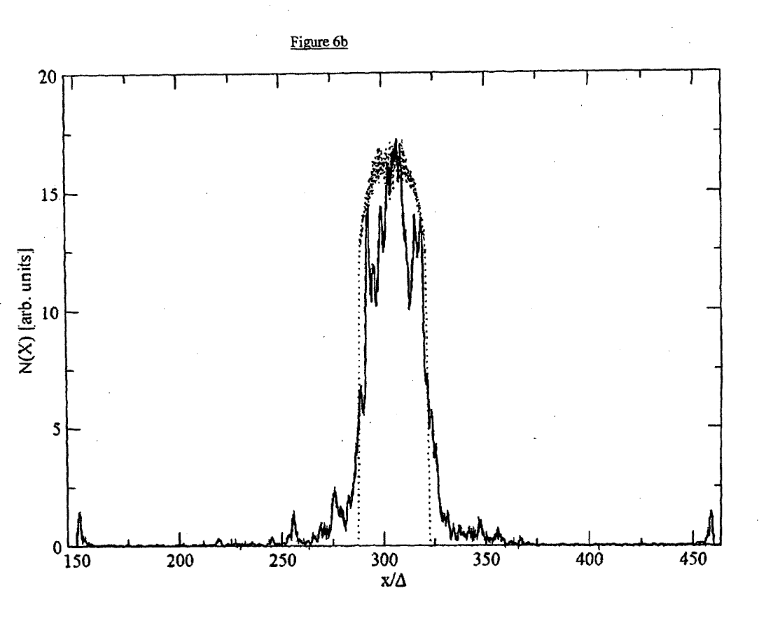

Thus, the general dynamics of the electron component can be described as an oscillatory motion around the target. The return time or the period of oscillations depends on the initial position x₀ of the fluid element. Electrons that initially are closer to the boundary of the plasma slab ((½−x₀)→0) have longer return times. The presence of this asynchronicity in the electron fluid motion quickly leads to “mixing” of the initially (set by the initial conditions) “ordered” electron trajectories. After a few tens of plasma period cycles, the electron phase space and density distributions evolve in such a way that the majority of electrons can be localized around the target, considerably shielding its charge. FIG. 6 shows the phase-space (a) and density (b) distributions of electrons at time t=150/ωₚₑ obtained from one-dimensional PIC simulations. As mentioned earlier, the initial condition for the electron momentum distribution was p_{e,0}(x)=sign(x)θ(½−|x|)10mₑc. The late time phase-space distribution shows the formation of an electron cloud concentric with the expanding ion layer having a rather broad momentum distribution. An electron structure appears at a distance from the target propagating away from it with velocity nearly equal to v_{e,0}. These can be the particles that have originated at a front of the electron cloud (|x₀|→½).

B. Coulomb explosion and the electric field structure beyond the target's surface. Without being bound by any particular theory of operation, the Coulomb explosion of the target, which leads to the gradual expansion of the ion layer, appears to render the ion density time-dependent. According to Eqn (4e), the change in ion density influences the longitudinal electric field profile. The electric field distribution (see Eqn (7)) calculated in the previous section can assume an infinite ion mass (χ=0). Therefore, in order to find out how the field structure changes with the expanding ion layer, the spatial and temporal evolution of ion density needs to be obtained. Its development can be governed by the action of the electric field inside the target. Under the assumption that the electrons have left the target, the self-consistent ion evolution can be found from the solution to the 1D ion hydrodynamic equations. As in the previous section, it can be advantageous to work in Lagrange representation, where the connection between both coordinates is expressed through the ion fluid element displacement:

x(x₀, t)=x₀+ξᵢ(x₀, t). (9)

The ion hydrodynamic equations in the Lagrange coordinates have the following form:

$$\begin{matrix}{\tilde{n}}_{i} ( {x}_{0} , t ) = {n}_{i} ( x , t ) = {\tilde{n}}_{i} ( {x}_{0} , 0 ) \frac{\partial {x}_{0}}{\partial x} & ( 10 a ) \\ \frac{{\partial }^{2} {ξ}_{i} ( {x}_{0} , t )}{\partial {t}^{2}} = \frac{{Z}_{i} e}{{m}_{i}} {\tilde{E}}_{in} ( {x}_{0} , t ) & ( 10 b ) \\ \frac{\partial {\tilde{E}}_{in}}{\partial {x}_{0}} = 4 π {eZ}_{i} {\tilde{n}}_{i} ( {x}_{0} , 0 ) , & ( 10 c )\end{matrix}$$

where Eᵢₙ denotes the electric field inside the target. For a flat initial density distribution ñᵢ(x₀, 0)=n₀θ(½−|x₀|), the solution of Eqs. (10) has the form:

$$\begin{matrix}{\tilde{E}}_{in} ( {x}_{0} , t ) = 4 π {en}_{o} {Z}_{i} {x}_{0} & ( 11 a ) \\ {ξ}_{i} ( {x}_{0} , t ) = χ \frac{{ω}_{pe}^{2}}{2} {t}^{2} {x}_{0} . & ( 11 b )\end{matrix}$$

As seen from Eqn (11a), the electric field vanishes in the middle of the target and linearly increases (in absolute value) away from it. Using Eqn (11b) and the relation (9) one can express the electric field and the ion density through the Euler variables (x, t) to give:

$$\begin{matrix}{n}_{i} ( x , t ) = \frac{{n}_{0}}{1 + \frac{{χω}_{pe}^{2} {t}^{2}}{2}} θ ( \frac{l}{2} - \frac{\vert x \vert}{1 + \frac{{χω}_{pe}^{2} {t}^{2}}{2}} ) & ( 12 a ) \\ {E}_{in} ( x , t ) = \frac{4 π {Z}_{i} {en}_{0} x}{1 + \frac{{χω}_{pe}^{2} {t}^{2}}{2}} , \vert x \vert \leq \frac{l}{2} ( 1 + \frac{{χω}_{pe}^{2} {t}^{2}}{2} ) & ( 12 b ) \\ {E}_{out} ( x , t ) = \pm 4 π {Z}_{i} {en}_{0} \frac{l}{2} , \vert x \vert > \frac{l}{2} ( 1 + \frac{{χω}_{pe}^{2} {t}^{2}}{2} ) & ( 12 c )\end{matrix}$$

Eqn (12a) describes the evolution of one-dimensional ion slab under the action of the Coulomb repulsive force (i.e., Coulomb explosion).

As described above, the simulation results indicate that the maximum kinetic energy of the accelerated protons can be determined by the structure of the longitudinal field beyond the surface of the target. Therefore, the spatio-temporal evolution of the electric field near the front of the expanding electron cloud is of interest. The initial conditions for these electrons can be x₀→½ and their displacement ξₑ(x₀, t) for ½<x₀+ξₑ(x₀, t) takes the following form:

$${ξ}_{e} ( {x}_{0} , t ) \approx {υ}_{e , 0} t - \frac{{ω}_{pe}^{2} {t}^{2}}{2 {( 1 + \frac{{p}_{e , 0}^{2}}{{m}_{e}^{2} {c}^{2}} )}^{3 / 2}} ( \frac{l}{2} - {x}_{0} ) . \qquad ( 13 )$$

Eqn (13) was obtained from the solution of Eqn (8b) in the limit ½−x₀→0 and together with the definition (Eqn (5)) constitutes the inversion procedure, which allows one to go back to Euler coordinates (x, t) and determine the electric field structure (in x, t coordinates) at the front of the electron cloud as presented in Bulanov, et al. The calculated field distribution however typically does not reflect the influence of the ion motion. In order to obtain the contribution of ions, the next order in the expansion of electric field in the smallness parameter χ can be obtained by substituting the density distribution function from Eqn (12a) into Eqn (6e):

$$\frac{\partial \tilde{E}}{\partial {x}_{0}} = 4 π {eZ}_{i} {n}_{0} [ \begin{matrix}\frac{1}{1 + \frac{{χω}_{pe}^{2} {t}^{2}}{2}} θ ( \frac{l}{2} - \frac{{x}_{0} + {ξ}_{e} ( {x}_{0} , t )}{1 + \frac{{χω}_{pe}^{2} {t}^{2}}{2}} ) \\ [ 1 + \frac{\partial {ξ}_{e} ( {x}_{0} , t )}{\partial {x}_{0}} ] - θ ( \frac{l}{2} - {x}_{0} )\end{matrix} ] , \\ for \frac{l}{2} < {x}_{0} + {ξ}_{e} ( {x}_{o} , t ) . \qquad ( 14 )$$

Using the Lagrange displacement for the electrons given by Eqn (13), Eqn (14) can be integrated to arrive at:

$$\tilde{E} ( {x}_{0} , t ) = 4 {πZ}_{i} {en}_{0} ( \frac{l}{2} - {x}_{0} - \frac{{υ}_{e , 0} t - \frac{l {ω}_{pe}^{2} {t}^{2}}{4 F}}{1 + \frac{{χω}_{pe}^{2} {t}^{2}}{2}} + C ( t ) ) ,$$

where F=(1+p_{e,0}²/mₑ²c²)^{3/2} and C(t) is an arbitrary function of time appearing as a result of indefinite integration. Its form can be found when χ=0 and the electric field can be provided by Eqn (7). The structure of the electric field at the front of electron cloud is:

$$\tilde{E} ( {x}_{0} , t ) = 4 {πZ}_{i} {en}_{0} ( \frac{l}{2} - {x}_{0} + \frac{( {υ}_{e , 0} t - \frac{l {ω}_{pe}^{2} {t}^{2}}{4 F} ) \frac{{χω}_{pe}^{2} {t}^{2}}{2}}{1 + \frac{{χω}_{pe}^{2} {t}^{2}}{2}} ) \qquad ( 15 )$$

The incorporation of the ion motion into the hydrodynamic description of both components renders the longitudinal electric field (at the front of expanding electron cloud) dependent on the physical parameters of the ions. The dependence is such that a larger value of the parameter χ results in larger electric field; for relativistic electrons v_{e,0}t>1wₚₑ²t²/(4F) for t<τ˜1000/ωₚₑ. This increase in the field strength typically leads to higher proton energy, which was also observed in the 2D PIC simulations (see FIG. 3). Note that Eqn (15) was obtained under the assumption that electrons do not return to the target. As discussed in the previous section, a majority of electrons will eventually come back, performing complicated oscillatory motion around the slab. The presence of these electrons will shield part of the total charge in the target, reducing its effective charge density. This leads to an overestimation of the contribution of ion motion, but its dependence on the physical characteristics of the target typically remains intact.

Using PIC simulations and a hydrodynamic analytical model, the proton acceleration during the interaction of petawatt laser pulses with double-layer targets has been investigated. The role the heavy ion slab plays in the efficiency of the proton acceleration can be quantitatively understood, and more specifically, the influence of the Coulomb explosion effect on the longitudinal electrostatic field. As electrons are expelled from the target, a strong electrostatic field can be generated in the region between the target's surface and the front of the expanding electron cloud. The spatial and temporal evolution of this field can be determined by both the ion dynamics inside the target (the Coulomb explosion) and the self-consistent electron dynamics outside of it. PIC simulation results indicate, that more robust ion expansion leads to more energetic protons. The simulated longitudinal electric field profile exhibits a trend in which a larger value of a parameter χ=Zᵢmₑ/mᵢ leads to larger values of the electric field in the region beyond the target's surface. This increase in the field strength typically leads to more energetic protons. In the examples described herein, up to 50% difference in the maximum proton energy was observed for the carbon substrate versus that made of platinum, even though they have the same ionization state. Using a simplified one-dimensional hydrodynamic model, the electric field profile at the front of the expanding electron cloud can be obtained. Taking into account the ion motion in the hydrodynamic description of electron-ion plasma results in an increase in the electric field strength in the region beyond the surface of the target. If there were no electrons present, the electric field inside the expanding ion target would typically be lower for substrates with larger values of the structural parameter χ, whereas its magnitude outside the target's surface would be the same, irrespective of the value of χ, as can be seen from Eqs. (12b, 12c). This would eventually lead to lower energies for the accelerated protons, which contradicts the simulation results as well as the analytical predictions. Thus, the observed increase in the magnitude of the electric field beyond the target's surface can be a result of the combined dynamics of both the ion and electron components.

As mentioned above, the ionization state of ions can be treated as a parameter, rather than a calculated value. On a qualitative level it can be feasible to ascertain that for a given laser intensity, the substrates with larger atomic masses can be ionized to higher ionization states. Whereas in order to quantitatively predict which substrate will maximize the proton energy, a reliable calculation method for the effective atomic ionization state is needed. In this respect, the work by Augst et al., Phys. Rev. Lett., 63, 2212, 1989, as carried out for noble gases, can be used as a possible starting point to further investigate other elements.

The methods provided herein can also be modified to account for collisional effects. The electron-ion collisions in the presence of laser light lead to inverse Bremsstrahlung heating of the electron component, introducing an extra mechanism for absorption of the light. Collisional effects can be important in the description of normal and anomalous skin effects, thus influencing the fraction of the laser light that gets transmitted through the target.

The dimensionality of the methods provided herein can also be modified. Two-dimensional PIC simulations can be quantitatively different from those in three-dimensional due to the difference in the form of the Coulomb interaction potential between the elementary charges (φ˜1nτ in 2D versus φ˜1/τ in 3D). One ramification that the maximum proton energy predicted by 2D methods can be overestimated compared to 3D methods. The predicted dependence of the maximum proton energy on the substrate structure parameter χ can also be determined by the dimensionality of the methods. Since both, 1D theoretical model and 2D simulations provide that the maximum proton energy depends on χ, this correlation is expected to be found in 3D methods.

The results of the modeling and simulation results provide methods for designing a laser-accelerated ion beam of the present invention. These methods include modeling a system including a heavy ion layer, an electric field, and high energy light positive ions having an energy distribution comprising a maximum light positive ion energy. Suitable modeling methods, such as PIC, are described above. Physical parameters of the heavy ion layer, the electric field, and the maximum light positive ion energy are then correlated using the modeling methods. The laser-accelerated ion beam is designed by varying the parameters of the heavy ion layer to optimize the energy distribution of the high energy light positive ions. Suitable methods for varying the parameters of the heavy ion layer, for example by simulation, are provided hereinabove.

Any type of target material can be used, and preferably the target comprises at least one material that gives rise to a heavy ion layer and one material that gives rise to a light ion material. In the targets and methods of various embodiments of the present invention, the heavy ion layer suitably comprises a material composed of atoms, ions, or a combination thereof, having an atomic mass greater than about that of the high energy light positive ions. Suitable heavy ion layers are derived from materials composed of atoms having a molecular mass greater than about 10 daltons, e.g., carbon, or any metal, or combination thereof. Examples of suitable metals for use in heavy ion layers of suitable targets include gold, silver, platinum, palladium, copper, or any combination there of. Suitable high energy light positive ions are derived from hydrogen, helium, lithium, beryllium, boron, carbon, nitrogen, or oxygen, fluorine, neon or argon, or any combination thereof. Protons are suitably prepared from hydrogen-containing matter composed of ions, molecules, compositions, or any combination thereof. Suitable hydrogen containing material can be formed as a layer adjacent to a metal layer of the target. In certain embodiments, the high energy light positive ions are produced from a layer of light atom rich material. Suitable light atom rich materials include any type of matter that is capable of keeping hydrogen, helium, lithium, beryllium, boron, carbon, nitrogen, or oxygen, fluorine, neon or argon, or any combination thereof, adjacent to or proximate to the heavy ion layer. Suitable examples of light atom rich materials include water, organic materials, noble gases, polymers, inorganic materials, or any combination thereof. In some embodiments the protons originate from a thin layer of hydrocarbons or water vapor present on the surface of the solid target. Any type of coating technology can be used in preparing targets. Suitable materials for providing the high energy light positive ions can be readily applied to one or more materials (e.g, substrates) composed of heavy atoms that give rise to the heavy ions.

In some embodiments multiple layers of light ion materials can be used. In other embodiments, materials that produce multiple ion types that can then be separated in the field can also be incorporated. For effective light ion acceleration, a very strong electric field is produced using a laser-pulse interaction with a high-density target material. Suitable laser pulses are in the petawatt range. In some embodiments, various materials composed of light ions can be used where the electron density in the material is high. In a sandwich-type target system different species of ions can be accelerated, which in turn can be separated by applying electric and magnetic fields, as described in further details in “High Energy Polyenergetic Ion Selection Systems, Ion Beam Therapy Systems, and Ion Beam Treatment Centers”, WO2004109717, International Patent Application No. PCT/US2004/017081, claiming priority to U.S. App. No. 60/475,027, filed Jun. 2, 2003, the portion of which pertaining to ion selection systems is incorporated by reference herein. Examples of methods of modulating laser-accelerated protons for radiation therapy that can be adapted for use in the present invention are described in further detail in “Methods of Modulating Laser-Accelerated Protons for Radiation Therapy”, WO2005057738, U.S. application Ser. No. ______, claiming priority to U.S. App. No. 60/475,027, filed Jun. 2, 2003, and U.S. App. No. 60/526,436, filed Dec. 2, 2003, the portion of which pertaining to methods of modulating laser-accelerated protons for radiation therapy is incorporated by reference herein.

The results of the modeling and simulation results also provide methods for designing targets used for generating laser-accelerated ion beams. These methods include the steps of modeling a system including a target, an electric field, and high energy light positive ions having an energy distribution comprising a maximum light positive ion energy. In these methods, the target includes a heavy ion layer characterized by a structural parameter χ. The structure parameter χ is defined as Zᵢmₑ/mᵢ, wherein Zᵢ is the specific ionization state of heavy ions in the heavy ion layer, mₑ is the mass of an electron, and mᵢ is the mass of the heavy ions in the heavy ion layer. The methods for designing targets in these embodiments include the step of varying the structural parameter χ that characterizes the target to optimize the energy distribution of the high energy light positive ions. The structural parameter χ can be varied in the range of from. about 10⁻⁶ to about 10⁻³, and in particular in the range of from about 10⁻⁵ to about 10⁻⁴. These values are particular useful in embodiments where the high energy light ions include protons. Values of the structural parameter can be selected by persons of ordinary skill in the art by the suitable selection of materials having knowledge of the specific ionization state of a particular heavy ion, the mass of an electron (about 9×10⁻³¹ kg) , and the mass of the particular heavy ion. Suitable high energy light positive ions can have an optimal energy distribution in most embodiments up to about 50 MeV, and in some embodiments even up to about 250 MeV.

The heavy ion layer suitably is derived from materials that include atoms having an atomic mass greater than about 10 daltons, examples of which include carbon, a metal, or any combination thereof. Suitable metals include gold, silver, platinum, palladium, copper, or any combination thereof. In some embodiments the high energy light positive ions comprise protons or carbon, or any combination thereof. Suitable high energy light positive ions are derived from hydrogen, helium, lithium, beryllium, boron, carbon, nitrogen, or oxygen, fluorine, neon or argon, or any combination thereof. Suitable high energy light positive ions can have an energy in the range of from about 50 MeV to about 250 MeV by adjusting both the electric field strength through selection of a suitably intense petawatt laser pulse and the value of the structural parameter χ of the target material. Protons are suitably prepared from hydrogen-containing matter composed of ions, molecules, compositions, or any combination thereof. Suitable hydrogen-containing materials can be formed as a layer adjacent to a metal layer of the target.

The results of the modeling and simulation results also provide targets that are useful for generating laser-accelerated high energy light positive ion beams in a system. Targets according to this embodiment of the present invention can be designed by the process of modeling a system including the target, an electric field, and high energy light positive ions having an energy distribution comprising a maximum light positive ion energy. In these embodiments, the target includes a heavy ion layer characterized by the structural parameter χ as defined above. The method includes varying the structural parameter χ to optimize the energy distribution of the high energy light positive ions. The structural parameter χ can be varied iteratively or through PIC simulations for optimizing the energy distributions. Suitable materials can be selected for controlling the structural parameter χ as described above.

The results of the modeling and simulation results also provide targets that are useful for generating laser-accelerated ion beams in a system that includes a target, an electric field, and high energy light positive ions. Suitable high energy positive ions generated with this system will have an energy distribution that includes a maximum light positive ion energy. Suitable targets in these systems will include a heavy ion layer characterized by a structural parameter χ, wherein varying the structural parameter χ maximizes the energy distribution of the high energy light positive ions of the modeled system. Selection of the structural parameter χ and the selection of materials is described above.

In various embodiments, combinations of heavy atom containing materials and light atom materials can be used to provide, respectively, the heavy ions and the light ions for preparing the targets. For example, one embodiment is a double layer target comprising a light atom layer composed of a hydrocarbon (e.g., carbon and protons) and a heavy atom layer composed of metals, for example gold or copper. In one embodiment, high-quality (e.g., high energy, low energy spread in a distribution, low emittance) light ion beams can be produced using a sandwich-like target system. Such a sandwich-like target system can include a first layer substrate having a high electron density, not infinitesimal value for the structural parameter χ comprising the heavier atoms. In these embodiments, the second layer, which comprises light atoms that give rise to the high energy light ions, should be much thinner than the first layer substrate. Interaction of an intense laser pulse with such a target geometry gives rise to acceleration of the light ions, as described above, to form a high energy light ion beam. As mentioned above, a wide variety of light ions can be accelerated using this techniques.

Polymers can also be used in designing suitable targets. Various types of polymers and plastic materials can be used in various embodiments. Any plastic material can be a good candidate for preparing targets according to the present invention. Plastic materials, which are composed of polymer molecules of carbon, hydrogen, oxygen, nitrogen, sulfur, phosphorus atoms, and any combination thereof, are suitably dense enough to produce high electron concentration after ionization by the laser. Suitable light ions have low masses and give rise a finite value of the structural parameter χ.

Some embodiments are capable of designing targets that generate a high energy light ion beam composed of high energy carbon ions. For example, a sandwich-like target for accelerating carbon ions can be produced by coating a metal substrate with a carbon layer having a thickness in the range of from about 50 nm to about 100 nm. Suitable metal substrates include metal foils, such as copper, gold, silver, platinum and palladium, and the like.

Various additional embodiments are envisioned in which the parameters of different layers can be calculated. For example, a reliable model can be provided for predicting ion charge state distribution in a substrate for a given laser-pulse characteristics. Other ways of optimizing the beam or target in addition to, or in complement with, PIC simulations can also be carried out. For example, in one embodiment, the laser pulse shape can be modified with a prepulse (e.g., the laser pedestal), which precedes the main pulse. The laser prepulse is intense enough to dramatically change the shape and the physical condition of the main substrate, so that when the main laser pulse arrives at the target, it interacts with the substrate of altered characteristics. Accordingly, modeling of the laser-prepulse interaction with the target in conjunction with PIC simulations (together with reliable ionization model for the substrate) can give rise to an even more accurate understanding of the physical processes occurring. Inclusion of the results of the prepulse effects can aid in the development of improved target design and methods of synthesizing high energy light ion beams.

In additional embodiments, it is envisioned that this method can be used to design various targets and give rise to synthesizing high energy light ion beams. Combining hydrodynamic and PIC simulations as described herein gives rise to the light-ion energy spectrum for the given initial laser pulse and target properties. Routine experimentation by those of skill in the art in conducting parametric studies of different target materials, shapes and dimensions can yield additional optimal laser/target characteristics that will give rise to high quality accelerated light ions suitable for hadron therapy for the treatment of cancer and other diseases.

What is claimed:

1. A method for designing a laser-accelerated ion beam, comprising:
modeling a system including a heavy ion layer, an electric field, and high energy light positive ions having an energy distribution comprising a maximum light positive ion energy;
correlating physical parameters of the heavy ion layer, the electric field, and the maximum light positive ion energy using said model; and
varying the parameters of the heavy ion layer to optimize the energy distribution of the high energy light positive ions.

2. The method according to claim 1, wherein the heavy ion-layer comprises carbon.

3. The method according to claim 1, wherein the heavy ion layer comprises a metal, or any combination of metals.

4. The method according to claim 3, wherein the metal comprises gold, silver, platinum, palladium, copper, or any combination there of.

5. The method according to claim 1, wherein the high energy light positive ions are derived from hydrogen, helium, lithium, beryllium, boron, carbon, nitrogen, or oxygen, fluorine, neon or argon, or any combination thereof.

6. The method according to claim 1, wherein the high energy light positive ions are produced from a layer of light positive ion rich material.

7. The method according to claim 6, wherein the light positive ion rich material comprises water, hydrocarbons, noble gases, polymers, an inorganic material, or any combination thereof.

8. A method for designing a target used for generating laser-accelerated ion beams, comprising:
modeling a system including a target, an electric field, and high energy light positive ions having an energy distribution comprising a maximum light positive ion energy, said target comprising a heavy ion layer characterized by a structural parameter χ; and
varying the structural parameter χ to optimize the energy distribution of the high energy light positive ions.

9. The method according to claim 8, wherein the heavy ion layer comprises carbon.

10. The method according to claim 8, wherein the heavy ion layer comprises a metal, or any combination of metals.

11. The method according to claim 10, wherein the metal comprises gold, silver, platinum, palladium, copper, or any combination thereof.

12. The method according to claim 10, wherein the metal comprises copper.

13. The method according to claim 8, wherein the high energy light positive ions comprise protons or carbon, or any combination thereof.

14. The method according to claim 8, wherein the high energy light positive ions are produced from a layer of light positive ion rich material.

15. The method according to claim 14, wherein the light positive ion rich material comprises water, hydrocarbons, noble gases, or polymers, or any combination thereof.

16. A target used for generating laser-accelerated high energy light positive ion beams in a system, said target made by the process of:
modeling a system including the target, an electric field, and high energy light positive ions having an energy distribution comprising a maximum light positive ion energy, said target comprising a heavy ion layer characterized by a structural parameter χ; and
varying the structural parameter χ to optimize the energy distribution of the high energy light positive ions.

17. The target made by the process of claim 16, wherein the heavy ion layer comprises carbon.

18. The target made by the process of claim 16, wherein the heavy ion layer comprises a metal, or any combination of metals.

19. The target made by the process of claim 18, wherein the metal comprises gold.

20. The target made by the process of claim 18, wherein the metal comprises copper.

21. The target made by the process of claim 16, wherein the high energy light positive ions comprise protons or carbon, or any combination thereof.

22. The target made by the process of claim 16, wherein the high energy light positive ions are produced from a layer of light positive ion rich material.

23. The target made by the process of claim 22, wherein the light positive ion rich material comprises water, hydrocarbons, noble gases, or polymers, or any combination thereof.

24. A target used for generating laser-accelerated ion beams in a system including the target, an electric field, and high energy light positive ions having an energy distribution comprising a maximum light positive ion energy, said target comprising:
a heavy ion layer characterized by a structural parameter χ, wherein varying the structural parameter χ maximizes the energy distribution of the high energy light positive ions of the modeled system.

25. The target made by the process of claim 24, wherein the heavy ion layer comprises carbon.

26. The target made by the process of claim 24, wherein the heavy ion layer comprises a metal, or any combination of metals.

27. The target made by the process of claim 26, wherein the metal comprises gold.

28. The target made by the process of claim 26, wherein the metal comprises copper.

29. The target made by the process of claim 24, wherein the high energy light positive ions comprise protons or carbon, or any combination thereof.

30. The target made by the process of claim 24, wherein the high energy light positive ions are produced from a layer of light positive ion rich material.

31. The target made by the process of claim 30, wherein the light positive ion rich material comprises water, hydrocarbons, noble gases, polymers, or any combination thereof.

32. The method according to claim 8, wherein the structural parameter χ is defined as Zᵢmₑ/mᵢ, wherein Zᵢ is the specific ionization state of heavy ions in the heavy ion layer, mₑ is the mass of an electron, and mᵢ is the mass of the heavy ions in the heavy ion layer.

33. The method according to claim 32, wherein the structural parameter χ has a value in the range of from about 10⁻⁶ to about 10⁻³.

34. The method according to claim 33, wherein the structural parameter χ has a value in the range of from about 10⁻⁵ to about 10⁻⁴.

35. The target according to claim 16, wherein the structural parameter χ is defined as Zᵢmₑ/mᵢ, wherein Zᵢ is the specific ionization state of heavy ions in the heavy ion layer, mₑ is the mass of an electron, and mᵢ is the mass of the heavy ions in the heavy ion layer.

36. The method according to claim 35, wherein the structural parameter χ has a value in the range of from about 10⁻⁶ to about 10⁻³.

37. The method according to claim 36, wherein the structural parameter χ has a value in the range of from about 10⁻⁵ to about 10⁻⁴.

38. The target according to claim 24, wherein the structural parameter χ is defined as Zᵢmₑ/mᵢ, wherein Zᵢ is the specific ionization state of heavy ions in the heavy ion layer, mₑ is the mass of an electron, and mᵢ is the mass of the heavy ions in the heavy ion layer.

39. The method according to claim 38, wherein the structural parameter χ has a value in the range of from about 10⁻⁶ to about 10⁻³.

40. The method according to claim 39, wherein the structural parameter χ has a value in the range of from about 10⁻⁵ to about 10⁻⁴.

41. The method of claim 1, wherein the maximum light positive ion energy is in the range of from about 50 MeV to 250 MeV.

42. The method of claim 8, wherein the maximum light positive ion energy is in the range of from about 50 MeV to 250 MeV.

43. The target of claim 16, wherein the maximum light positive ion energy is in the range of from about 50 MeV to 250 MeV.

44. The target of claim 24, wherein the maximum light positive ion energy is in the range of from about 50 MeV to 250 MeV.
